(19) United States
(12) Patent Application Publication
(10) Pub. No.: US 2024/0158435 A1
(43) Pub. Date: May 16, 2024

(54) BIOCATALYTICAL COMPOSITION AND USE FOR GENERATION OF IMMUNOLIGAND-PAYLOAD CONJUGATES

(71) Applicants: INOFEA AG, Muttenz (CH); NBE-THERAPEUTICS AG, Basel (CH)

(72) Inventors: Patrick SHAHGALDIAN, Hegenheim (FR); Manon BRIAND, Ennetbaden (CH); Philippe F.-X. CORVINI, Leymen (FR); Maria Rita CORRERO, Hegenheim (FR); Yves DUDAL, Zürich (CH); Ulf GRAWUNDER, Beckenried (CH); Rémy GEBLEUX, Basel (CH); Roger R. BEERLI, Adlikon (CH)

(21) Appl. No.: 18/282,361

(22) PCT Filed: Mar. 17, 2022
(86) PCT No.: PCT/EP2022/056952

§ 371 Date: Sep. 15, 2023

(30) Foreign Application Priority Data
Mar. 18, 2021 (EP) 21163418.3

(51) Int. Cl. C07K 1/107; C07K 1/34; C12N 9/64; C12N 11/14

(52) U.S. Cl. CPC C07K 1/1077; C07K 1/34; C12N 9/641; C12N 11/14; C07K 2319/22; C12Y 304/2207

(57) ABSTRACT

The present invention relates to a composition comprising a solid carrier, a capture moiety, a functional protein, a first linker connecting the capture moiety to the solid carrier, a second linker connecting the functional protein to the capture moiety, a first protective layer fully embedding the solid carrier, fully or partially embedding the first linker, and not or partially embedding the capture moiety, a second protective layer fully or partially embedding the capture moiety, fully embedding the second linker, and fully or partially embedding the functional protein.

### CROSS-REFERENCE TO RELATED APPLICATIONS

The present application is the U.S. National Phase of PCT/EP2022/056952, filed on 17 Mar. 2022, which claims priority to European Patent Application No. 21163418.3, filed on 18 Mar. 2021, the entire contents of which are incorporated herein by reference.

### THE FIELD OF THE INVENTION

The present invention relates to a composition comprising a solid carrier; a capture moiety; a functional protein; a first linker connecting the capture moiety to the solid carrier; a second linker connecting the functional protein to the capture moiety; a first protective layer fully embedding the solid carrier, fully or partially embedding the first linker and not or partially embedding the capture moiety; and a second protective layer fully or partially embedding the capture moiety, fully embedding the second linker and fully or partially embedding the functional protein. The present invention also relates to methods of producing the composition, as well as methods of producing an immunoligand/payload conjugate by means of the composition.

### BACKGROUND OF THE INVENTION

Processes using proteins such as enzymes in industrial applications face the challenge that the employed enzymes may be exposed to harsh conditions, including shear forces, foaming, and/or unfavourable pH conditions and/or that the functional proteins or enzymes cannot be recovered from the process for being re-utilized in manufacturing, once they have been added in a batch manufacturing process. These challenges apply to wide range of uses of enzymes in industrial applications, including but not limited to fermentation processes, waste-management processes, food manufacturing processes and also in the manufacturing of therapeutic molecules. One solution to overcome these challenges is to immobilize functional proteins, or preferably enzymes, on a solid carrier and to shield the functional protein or enzyme by a protective molecular layer providing protection to various types of stresses (“shielding” the enzyme/protein), as disclosed in the prior art, e.g., in WO2015/014888. An additional benefit of such immobilized and protected (“shielded”) enzymes is that the immobilized enzymes on a carrier can be separated from an enzymatic process by various separation techniques, including, but not limited to centrifugation, filtration, diafiltration, membrane separation of the carrier from the soluble components of a manufacturing process. WO2015/014888 discloses a biocatalytical composition comprising a solid carrier, a functional protein like an enzyme and a protective layer for protecting the enzyme or functional protein by embedding the enzyme or functional protein at least partially and a process to produce such biocatalytical composition. It has further been shown in the prior art, that this process can be utilized to immobilize enzymes that can catalyse the enzymatic conjugation of a fluorescent dye to large proteins or molecules, for instance, but not limited to antibodies (Briand et al. (2020) Chem Commun. 56: 5170-5173).

However, the process and the composition described in WO2015/014888 and utilized by Briand et al. have the drawback that they lead to diminished catalytic activity in comparison to soluble enzymes utilized in the same reaction (Briand et al. (2020) Chem Commun. 56: 5170-5173). This has been observed for a variety of challenging enzymatic reactions, e.g., but not limited to the enzymatic manufacturing of an antibody-dye conjugate as an example for an immunoligand-payload conjugate of a large protein with a small molecule. These challenges may generally apply to protein glycosylations, protein digestions, and other biomolecule modification. Optimal enzymatic activity is of particular interest for enzymatic manufacturing of therapeutic immunoligand-payload conjugates, including, but not limited to manufacturing of antibody drug conjugates (ADCs), because of the high cost of goods of manufacturing of such immunoligand-payload conjugates their manufacturing intermediates and the catalytic enzymes that need to be manufactured under current Good Manufacturing Practice (cGMP), if the immunoligand-payload conjugates are to be used clinically for the treatment of patients. For this reason, there is a need for providing immobilized and “shielded” functional proteins or enzymes with optimal functional or enzymatic activity. Furthermore, enzymes immobilized on a solid carrier preserving optimal enzymatic activity can be re-used for repeated immunoligand-payload and ADC manufacturing processes to improve the cost of goods of manufacturing, because immobilized enzymes can be separated from the desired product during or after the conjugation process has been accomplished.

### SUMMARY OF THE INVENTION

The present invention provides a composition comprising a solid carrier; a capture moiety; a functional protein; a first linker connecting the capture moiety to the solid carrier; a second linker connecting the functional protein to the capture moiety; a first protective layer fully embedding the solid carrier, fully or partially embedding the first linker and not or partially embedding the capture moiety; and a second protective layer fully or partially embedding the capture moiety, fully embedding the second linker and fully or partially embedding the functional protein.

The present invention provides also a method of producing the composition, wherein the method includes:
(a) connecting a capture moiety to a solid carrier by a first linker;
(b) forming a first protective layer on the surface of the solid carrier fully embedding the solid carrier, fully or partially embedding the first linker, and not or partially embedding the capture moiety;
(c) connecting a functional protein to the capture moiety by a second linker; and
(d) forming a second protective layer on the surface of the first protective layer fully or partially embedding the capture moiety, fully embedding the second linker, and fully or partially embedding the functional protein.

Furthermore, the present invention provides a method of producing an immunoligand/payload conjugate, which method comprises conjugating (e.g., carrying out a conjugation reaction of) a payload to an immunoligand by means of the composition.

In bioconjugation fusing an antibody and a small molecular weight dye, the enzymatic activity of the composition of the present invention, with a two-layered immobilization of the enzyme showed a surprisingly better bioconjugation as compared to a conventionally immobilized enzyme with one layer and was essentially identical to the activity of the soluble enzyme. This demonstrates that the novel two layered immobilization process results in a directionally immobilized protein or enzyme, which leads to a functional or enzymatic composition with activity essentially identical to the corresponding soluble functional protein or enzyme.

### BRIEF DESCRIPTION OF THE DRAWINGS

FIG. 1 shows a schematic view of the site-specific immobilization of a functional protein.

FIG. 2 shows non-specific adsorption of sortase on the particles surface after growing layers with different composition. The layers were made of 3-aminopropyl-triethoxysilane (A), orthosilicate (T), and hydroxymethyltri-ethoxysilane (H) (layer designated “ATH”); 3-aminopropyl-triethoxysilane (A) and orthosilicate (T) (layer designated “AT”); and 3-aminopropyl-triethoxysilane (A), orthosilicate (T) and propyltriethoxysilane (P) (layer designated “ATP”), respectively. The layer made of ATH shows the lowest value of sortase non-specific adsorption.

FIG. 3A shows fluorescence scanning of SDS-PAGE gels of Sortase A covalently immobilized on silica particles and partially shielded with an organosilica layer (AT) and Sortase A immobilized on silica particles and shielded with double layer with different composition (AT-AT and ATH-AT).

FIG. 3B shows bioconjugation of an antibody to a fluorescent probe catalysed by sortase particles. Sortase A covalently immobilized on silica particles and partially shielded with an organosilica layer (AT). Sortase A immobilized on silica particles and shielded with double layer with different composition (AT-AT and ATH-AT).

FIG. 4A and FIG. 4B show kinetics of conjugation of a FITC dye to an antibody heavy and light chain as visualized by UV-irradiation. Shown is the sortase-enzyme mediated conjugation at the ca. 50 kD heavy and at the ca. 30 kD light chains of the antibody after the time-points indicated on top of the SDS-PAGE gel in which the conjugation to antibody heavy and light chains is visualized by UV-irradiation that leads to green fluorescence of the attached FITC dye (here shown in black bands). Panel of FIG. 4A shows the kinetics of conjugation with immobilized and shielded sortase A enzyme. Panel of FIG. 4B shows the kinetics of conjugation with equivalent amount of soluble enzyme.

FIG. 5A shows the yield of ADCs production in the bioconjugation of an antibody to a toxic payload catalysed by sortase particles or by soluble sortase. Sortase A covalently immobilized on silica particles and shielded with double layer (ATH-AT).

FIG. 5B shows the Drug Antibody Ratio (DAR) in the bioconjugation of an antibody to a toxic payload catalysed by sortase particles or by soluble sortase. Sortase A covalently immobilized on silica particles and shielded with double layer (ATH-AT).

FIG. 6A shows the yield of ADCs production in the bioconjugation of an antibody to a toxic payload catalysed by sortase particles. Sortase A covalently immobilized on silica particles and shielded with double layer (ATH-AT). Sortase A covalently immobilized on silica particles via glutaraldehyde crosslinking and shielded with an organosilica single layer (AT).

FIG. 6B shows the Drug Antibody Ratio (DAR) in the bioconjugation of an antibody to a toxic payload catalysed by sortase particles. Sortase A covalently immobilized on silica particles and shielded with double layer (ATH-AT). Sortase A covalently immobilized on silica particles via glutaraldehyde crosslinking and shielded with an organosilica single layer (AT).

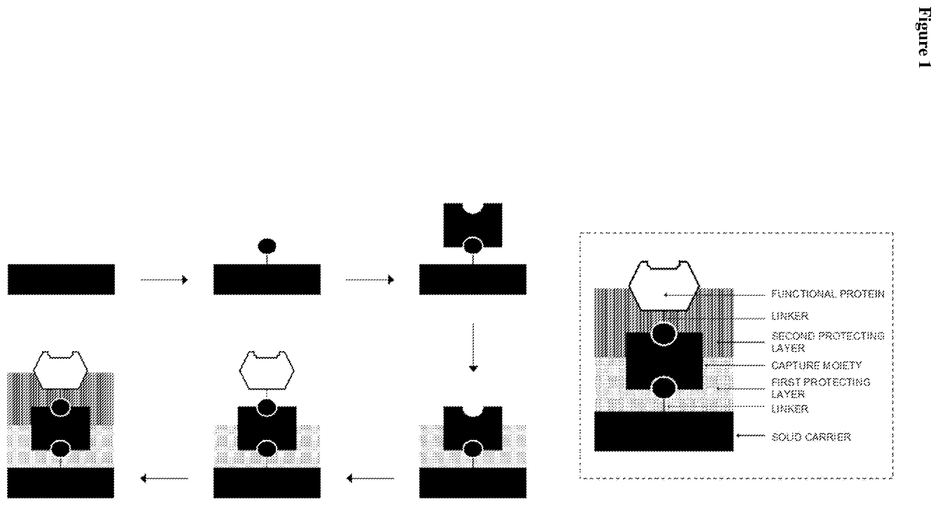

### DETAILED DESCRIPTION OF THE INVENTION

The present invention relates to a composition comprising a solid carrier; a capture moiety; a functional protein; a first linker connecting the capture moiety to the solid carrier; a second linker connecting the functional protein to the capture moiety; a first protective layer fully embedding the solid carrier, fully or partially embedding the first linker and not or partially embedding the capture moiety; and a second protective layer fully or partially embedding the capture moiety, fully embedding the second linker and fully or partially embedding the functional protein.

For the purposes of interpreting this specification, the following definitions will apply and whenever appropriate, terms used in the singular will also include the plural and vice versa. It is to be understood that the terminology used herein is for the purpose of describing particular embodiments only and is not intended to be limiting.

The term “solid carrier” as used herein refers usually to a particle. Preferably the solid carrier is a monodisperse particle or a polydisperse particle, more preferably a monodisperse particle. The solid carrier usually comprises organic particles, inorganic particles, organic-inorganic particles, self-assembling organic particles, silica particles, gold particles, titanium particles and is preferably a silica particle, more preferably a silica nanoparticle (SNP). The particle size of the solid carrier is usually between 1 nm and 1000 μm, preferably between 10 nm and 100 μm, particularly between 50 nm and 50 μm, more particularly between 100 nm and 1 μm.

The term “functional protein” as used herein refers to a protein which after being added to the solid carrier retains its characteristic, functional property. A functional protein in the sense of the present invention can be, e.g., an enzyme, an antibody, or RNA which has catalytic activity. Thus, in case the “functional protein” is an enzyme, which is the preferred functional protein, the carriers comprising the enzyme are enzymatically active. The functional protein is usually immobilized to the solid carrier, e.g., the functional protein is usually covalently and/or non-covalently bound to the solid carrier. The term “functional protein” as used herein comprises also a fragment of a functional protein, e.g., the functional protein may be an enzyme or a fragment thereof. A fragment of a functional protein as defined herein does usually have the same functional properties as the functional protein from which it is derived. The term “the functional protein from which it is derived” in relation to a fragment refers to the full-length functional protein from which the fragment is derived. The term “same functional properties as the functional protein from which it is derived” refers to the molecular function (or one of the molecular functions) of the full-length protein from which the fragment is derived, which for example can be an enzymatic activity.

The term “linker” as used herein refers to any linking reagents containing groups, which are capable of binding to specific functional groups (e.g., primary amines, sulfhydryls, etc.) such that, in the case of the first linker, one end of the linker binds to the surface of the carrier material and the other end to the capture moiety and, in case of the second linker, one end of the linker binds to the capture moiety and the other end binds or may be fused to the functional protein. Various types of linkers are known in the art, including but not limited to straight or branched-chain carbon linkers, heterocyclic carbon linkers, peptide linkers, polyether linkers, and linkers that are known in the art as tags. For example, a first linker may be immobilized on a solid carrier, e.g., on the silica surface as a carrier material and then a capture moiety may be bound to an unoccupied binding-site of the linker. Alternatively, the first linker may firstly bind to a capture moiety and then the linker bound to a capture moiety may bind with its unoccupied binding-site to the solid carrier, e.g., to the silica surface as a carrier material. A second linker may be bound to the capture moiety and then the functional protein may be bound to an unoccupied binding-site of the linker. Alternatively, the second linker may firstly bind or may be fused to a functional protein and then the linker bound or fused to a functional protein may bind with its unoccupied binding-site to the capture moiety. Preferably, if the second linker is a tag, the tag is preferably fused to a functional protein, preferably to the C-terminus of a functional protein. A tag fused to the functional protein binds to the tag binding-site of the capture moiety, usually such that the functional protein is immobilized in a directional manner.

The term “tag” as used herein, may encompass affinity tags, solubilization tags, chromatography tags and epitope tags. Affinity tags (also used as purification tags) are appended/fused to proteins so that they allow purification of the tagged molecule from their crude biological source using an affinity purification techniques. These include chitin-binding protein (CBP), maltose binding protein (MBP), glutathione-S-transferase (GST), biotin and modified biotin. The poly(His) tag, preferably a 6×His tag, is a widely-used tag; it binds to metal-containing matrices. Solubilization tags are used, especially for recombinant proteins expressed in chaperone-deficient species such as E. coli, to assist in the proper folding in proteins and keep them from precipitating. These include thioredoxin (TRX) and poly(NANP). Some affinity tags have a dual role as a solubilization agent, such as MBP, and GST.

Preferably, the linker, i.e., the first and/or second linker, in particular the first and second linker is a tag, preferably a tag selected from the group consisting of protein purification tags and affinity tags. Protein purification tags and affinity tags are usually selected from the group consisting of Protein A, LacZ, His-tag, Glutathione-S-Transferase, Maltose-Binding Protein, Calmodulin-Binding Peptide, Intein-Chitin Binding Domain, His-Patch ThioFusion, Epitope Tags, and Streptavidin- and Biotin-based Tags such as Strep-tag, Twin-Strep tag®, biotin and modified biotin (for a review of protein purification tags and affinity tags see Kimple et al, Current Protocols in Protein Science 9.9.1-9.9.23 Aug. 2013, DOI:10.1002/0471140864.ps0909s73). More preferably, the linker, i.e., the first and/or second linker, in particular the first and second linker are selected from the group consisting of a Strep-tag, a Twin-Strep tag®, biotin and modified biotin. The term “Strep-tag” refers to a synthetic peptide consisting of at least eight amino acids (Trp-Ser-His-Pro-Gln-Phe-Glu-Lys). This peptide sequence exhibits intrinsic affinity towards engineered streptavidins such as, e.g., Strep-Tactin®. The term “Twin-Strep tag® refers to a synthetic peptide consisting of two times at least eight amino acids (Trp-Ser-His-Pro-Gln-Phe-Glu-Lys). As a Strep-tag this peptide sequence exhibits intrinsic affinity towards engineered streptavidins such as, e.g., Strep-Tactin®. The term “modified biotin” as used herein refers to engineered variants of biotin, i.e., functional variants with a modified structure which bind to streptavidin or an engineered streptavidin like Sulfo-NHS-Biotin.

The term “capture moiety” as used herein refers to any molecule that can bind to or be bound to a solid carrier of interest. The capture moiety is usually bound, preferably non-covalently bound to the solid carrier via the first linker. A suitable capture moiety of the present invention is, e.g., an oligonucleotide; a polypeptide; an antibody or a functional fragment thereof; an avidin; an engineered avidin; a streptavidin; an engineered streptavidin, an aptamer; Spiegelmer™ (a L-RNA aptamer); a glutathione; or an S-peptide. The term “engineered avidin” and “engineered streptavidin “as used herein refers to engineered variants of avidin and streptavidin respectively, i.e., functional variants with a modified structure which bind to biotin. A preferred engineered streptavidin is Strep-Tactin®. Strep-Tactin® binds avidly to the peptide Strep-tag and biotin, respectively, in a manner comparable to streptavidin binding to biotin.

The term “protective layer” as used herein refers to a layer for protecting the functional properties of a functional protein or enzyme. The first protective layer covers fully the solid carrier in order to avoid non-specific adsorption of the functional protein at the surface of the solid carrier. The second protective layer covers fully or partially the functional protein to support the correct orientation of the functional protein. The layer, i.e., the first and/or second protective layer of the present invention is usually built with building blocks at least part of which are monomers capable of interacting with both each other usually by covalent binding and the immobilized functional protein usually by non-covalent binding. The first and the second protective layer can consist of the same or different material, e.g., of the same or different building blocks. Thus, in one embodiment the first and the second protective layer consist of the same material, e.g., of the same building blocks. Preferably, the first and the second protective layer consist of different material, e.g., of different building blocks. The protective layers are usually homogeneous layers preserving the properties of functional proteins or preferably enzymes embedded in the protective layer. The protective layers are usually homogeneous layers where at least 50%, preferably at least 70%, more preferably at least 90% of the functional proteins, or preferably enzymes embedded in the protective layer are orientated in the same direction (i.e., immobilized in a directional manner).

The term “first protective layer fully embedding the solid carrier” or “fully embedding the solid carrier” which both are used synonymously herein shall mean that the solid carrier is fully covered by the first protective layer.

The term “first protective layer fully or partially embedding the first linker” or “fully or partially embedding the first linker” which both are used synonymously herein, shall mean that the first linker is partially or fully covered by the first protective layer, thus, the first linker is partially or fully embedded in the first protective layer. Preferably, the first linker is fully embedded in the first protective layer.

The term “first protective layer not or partially embedding the capture moiety” as used herein shall mean that the capture moiety is not covered at all or not fully covered by the first protective layer, thus, the capture moiety is not embedded or not fully embedded in the first protective layer. In one embodiment of the invention at least 10% of the capture moiety is covered by the first protective layer, though typically at least 50% will be covered. In a preferred embodiment, at least 30%, preferably at least 40%, more preferably at least 50%, in particular at least 60% of the capture moiety is covered by the first protective layer. In a particularly preferred embodiment around 30%, preferably around 40%, more preferably around 50%, in particular around 60% of the capture moiety is covered by the first protective layer.

The term “second protective layer fully or partially embedding the capture moiety” as used herein shall mean that the capture moiety is fully or partially covered by the second protective layer, thus, the capture moiety is fully or partially embedded in the protective layer. Preferably, the second protective layer is partially embedding the capture moiety. The coverage of the capture moiety by the second protective layer depends on the coverage of the capture moiety by the first protective layer, e.g., if 10% of the capture moiety are covered by the first protective layer 90% of the capture moiety is covered by the second protective layer or if 50% of the capture moiety is covered by the first protective layer 50% of the capture moiety are covered by the second protective layer. Usually around 90% of the capture moiety are covered by the first protective layer and around 10% of the capture moiety is covered by the second protective layer, around 80% of the capture moiety are covered by the first protective layer and around 20% of the capture moiety is covered by the second protective layer, around 70% of the capture moiety are covered by the first protective layer and around 30% of the capture moiety is covered by the second protective layer, around 60% of the capture moiety are covered by the first protective layer and around 40% of the capture moiety is covered by the second protective layer, around 50% of the capture moiety are covered by the first protective layer and around 50% of the capture moiety is covered by the second protective layer, around 40% of the capture moiety are covered by the first protective layer and around 60% of the capture moiety is covered by the second protective layer, around 30% of the capture moiety are covered by the first protective layer and around 70% of the capture moiety is covered by the second protective layer, around 20% of the capture moiety are covered by the first protective layer and around 80% of the capture moiety is covered by the second protective layer, around 10% of the capture moiety are covered by the first protective layer and around 90% of the capture moiety is covered by the second protective layer. In a particular embodiment, around 50% of the capture moiety is covered by the first protective layer and around 50% of the capture moiety is covered by the second protective layer.

The term “second protective layer fully embedding the second linker” or “fully embedding the second linker” which both are used synonymously herein shall mean that the second linker is fully covered by the second protective layer.

The term “second protective layer fully or partially embedding the functional protein” or “fully or partially embedding the functional protein” which both are used synonymously herein shall mean that the functional protein is partially or fully covered by the second protective layer, thus, the functional protein is partially or fully embedded in the second protective layer. Preferably the functional protein is partially embedded.

The term “partially embedded functional protein” as used herein shall mean that the functional protein is not fully covered by the second protective layer, thus, the functional protein is not fully embedded in the protective layer. In one embodiment less than 50% of the functional protein of interest are covered by the protective layer, though typically more at least 70% will be covered, thus improving protection of the functional protein. In a preferred embodiment, at least 70%, more preferably at least 80%, even more preferably at least 90%, most preferably at least 95% of the functional protein of interest is covered by the protective layer. In another preferred embodiment, around 70% to around 95%, more preferably around 80% to around 95%, even more preferably around 90% to around 95%, most preferably around 90% to around 95, 96, 97, 98 or 99% of the functional protein of interest are covered by the protective layer. In a particularly preferred embodiment, around 70%, particularly around 80%, more particularly around 90%, most particularly around 95% of the functional protein of interest are covered by the protective layer. In a more particularly preferred embodiment, around 70%, particularly around 80%, more particularly around 90%, most particularly around 95% of the functional protein of interest is covered by the protective layer, wherein the active site is not covered.

The term “fully embedded functional protein” as used herein shall mean that the functional protein of interest according to the invention is fully, i.e., 100% covered by the second protective layer, i.e., that also the active site is covered.

As used herein, the term “immunoligand” is meant to define an entity, an agent or a molecule that has affinity to a given target, e.g., a receptor, a cell surface protein, a cytokine or the like. Such immunoligand may optionally block or dampen agonist-mediated responses, or inhibit receptor-agonist interaction. Most importantly, however, the immonoligand may serve as a shuttle to deliver a payload to a specific site, which is defined by the target recognized by the immunoligand. Thus, an immunoligand targeting, for instance, but not limited to a receptor, delivers its payload to a site which is characterized by abundance of the receptor. Immunoligands include, but are not limited to, antibodies, antibody fragments, antibody-based binding proteins, antibody mimetics, receptors, soluble decoy receptors, scaffold proteins with affinity for a given target and ligands of receptors.

“Antibodies”, also synonymously called “immunoglobulins” (Ig), are generally comprising four polypeptide chains, two heavy (H) chains and two light (L) chains, and are therefore multimeric proteins, or an equivalent Ig homologue thereof (e.g., a camelid nanobody, which comprises only a heavy chain, single domain antibodies (dAbs) which can be either be derived from a heavy or light chain); including full length functional mutants, variants, or derivatives thereof (including, but not limited to, murine, chimeric, humanized and fully human antibodies, which retain the essential epitope binding features of an Ig molecule, and including dual specific, bispecific, multispecific, and dual variable domain immunoglobulins; Immunoglobulin molecules can be of any class (e.g., IgG, IgE, IgM, IgD, IgA, and IgY), or subclass (e.g., IgG1, IgG2, IgG3, IgG4, IgA1, and IgA2) and allotype.

An “antibody drug conjugate” (ADC), as used herein, relates to either an antibody, or an antibody fragment, or an antibody-based binding protein, coupled to a small molecular weight active pharmaceutical ingredient (API), including, but not limited to a toxin (including e.g., but not limited to, tubulin inhibitors, actin binders, RNA polymerase inhibitors, DNA-intercalating and modifying/damaging drugs), a kinase inhibitor, or any API that interferes with a particular cellular pathway that is essential for the survival of a cell and/or essential for a particular physiologic cellular pathway.

An “antibody derivative or fragment”, as used herein, relates to a molecule comprising at least one polypeptide chain derived from an antibody that is not full length, including, but not limited to (i) a Fab fragment, which is a monovalent fragment consisting of the variable light (VL), variable heavy (VH), constant light (CL) and constant heavy 1 (CHI) domains; (ii) a F(ab′)2 fragment, which is a bivalent fragment comprising two Fab fragments linked by a disulfide bridge at the hinge region; (iii) a heavy chain portion of a Fab(Fa) fragment, which consists of the VHand CHI domains; (iv) a variable fragment (Fv) fragment, which consists of the VLand VHdomains of a single arm of an antibody, (v) a domain antibody (dAb) fragment, which comprises a single variable domain; (vi) an isolated complementarity determining region (CDR); (vii) a single chain FvFragment (scFv); (viii) a diabody, which is a bivalent, bispecific antibody in which VHand VLdomains are expressed on a single polypeptide chain, but using a linker that is too short to allow for pairing between the two domains on the same chain, thereby forcing the domains to pair with the complementarity domains of another chain and creating two antigen binding sites; and (ix) a linear antibody, which comprises a pair of tandem Fvsegments (VH-CH1-VH-CH1) which, together with complementarity light chain polypeptides, form a pair of antigen binding regions; and (x) other non-full length portions of immunoglobulin heavy and/or light chains, or mutants, variants, or derivatives thereof, alone or in any combination.

The term “modified antibody format”, as used herein, encompasses antibody-drug-conjugates, Polyalkylene oxide-modified scFv, Monobodies, Diabodies, Camelid Antibodies, Domain Antibodies, bi- or trispecific antibodies, IgA, or two IgG structures joined by a J chain and a secretory component, shark antibodies, new world primate framework+non-new world primate CDR, IgG4 antibodies with hinge region removed, IgG with two additional binding sites engineered into the CH3 domains, antibodies with altered Fc region to enhance affinity for Fc gamma receptors, dimerised constructs comprising CH3+VL+VH, and the like.

The term “antibody mimetic”, as used herein, refers to proteins not belonging to the immunoglobulin family, and even non-proteins such as aptamers, or synthetic polymers. Some types have an antibody-like beta-sheet structure. Potential advantages of “antibody mimetics” or “alternative scaffolds” over antibodies are better solubility, higher tissue penetration, higher stability towards heat and enzymes, and comparatively low production costs.

“Conjugation”, as used herein, relates to the covalent association of a molecule to another molecule by formation of a covalent bond.

The term “payload”, as used herein, represents any naturally occurring or synthetically generated molecule, including small-molecular weight molecules or chemical entities that can chemically be synthesized, and larger molecules or biological entities that need to be produced by fermentation of host cells and that confer a novel functionality to an immunoligand specific for binding to targets or antigens.

The term “toxin”, as used herein, means a cytotoxic compound of small molecular weight not exceeding a molecular weight of 2′500 Dalton that is cytotoxic to mammalian cells.

As used herein, the term “sequence-specific transpepeptidase” is meant to define a transpeptidase which needs at least one substrate peptide or protein with a given peptide sequence as recognition sequence (N-terminally and/or C-terminally) to connect the substrate peptide or protein to another peptide or protein, or a small-molecular weight compound containing a peptide or protein component.

As used herein, the term “site-specific transpepeptidase” is meant to define a transpeptidase which has a specific site in at least one substrate peptide or protein which it uses to conjugate to another peptide or protein, or a small-molecular weight compound containing a peptide or protein component.

As used herein, the term “radioactive agent” relates to an entity which has at least one atom with an unstable nucleus, and which is thus prone to undergo radioactive decay, resulting in the emission of gamma rays and/or subatomic particles such as alpha or beta particles, which have a cell killing effect. In the present context, radioactive agents are meant to impair, or even kill, pathogenic entity, e.g., a cancer cell.

The term “marker” (also called “detection tag”), as used herein, may refer to any molecule or moiety that comprises one or more appropriate chemical substances or enzymes, which directly or indirectly generate a detectable compound or signal in a chemical, physical or enzymatic reaction.

As used herein, the term “anti-inflammatory drug” relates to compounds that reduce inflammation. This can be, e.g., steroids, just like specific glucocorticoids (often referred to as corticosteroids), which reduce inflammation or swelling by binding to glucocorticoid receptors.

The term further encompasses non-steroidal anti-inflammatory drugs (NSAIDs), which counteract the cyclooxygenase (COX) enzyme. On its own, COX enzyme synthesizes prostaglandins, creating inflammation. In whole, the NSAIDs prevent the prostaglandins from ever being synthesized, reducing or eliminating the pain. The term further encompasses Immune Selective Anti-Inflammatory Derivatives (ImSAIDs), which are a class of peptides that alter the activation and migration of inflammatory cells, which are immune cells responsible for amplifying the inflammatory response. As used herein, the term “toxin” relates to a molecule which is toxic to a living cell or organism. Toxins may be peptides, or proteins or preferably small molecular weight compounds, that are meant to impair, or even kill, pathogenic entity, e.g., a cancer cell. Toxins, as meant herein, encompass, in particular, cellular toxins. Preferably, the toxin is a small molecular toxin, i.e., having a molecular weight of <2500 Da.

As used herein, the term “cytokine” refers to small cell-signaling protein molecules that are secreted by numerous cells and are a category of signaling molecules used extensively in intercellular communication. Cytokines can be classified as proteins, peptides, or glycoproteins; the term “cytokine” encompasses a large and diverse family of regulators produced throughout the body by cells of diverse embryological origin. In the present context, cytokines are for example meant to impair, or even kill, pathogenic entity, e.g., a cancer cell.

As used herein, the term “chemotherapeutic agent” relates to molecules that have the functional property of inhibiting a development or progression of a neoplasm, particularly a malignant (cancerous) lesion, such as a carcinoma, sarcoma, lymphoma, or leukemia. Inhibition of metastasis or angiogenesis is frequently a property of anti-cancer or chemo therapeutic agents. A chemo therapeutic agent may be a cytotoxic or chemo therapeutic agent. Preferably, the chemotherapeutic agent is a small molecular weight cytostatic agent, which inhibits or suppresses growth and/or multiplication of cancer cells.

In a first aspect, the present invention provides a composition comprising
a solid carrier;
a capture moiety;
a functional protein;
a first linker connecting the capture moiety to the solid carrier;
a second linker connecting the functional protein to the capture moiety;
a first protective layer fully embedding the solid carrier, fully or partially embedding the first linker, and not or partially embedding the capture moiety; and
a second protective layer fully or partially embedding the capture moiety, fully embedding the second linker, and fully or partially embedding the functional protein.

The solid carrier and the capture moiety can be connected by the first linker by non-covalent binding or covalent binding. The capture moiety and the functional protein can be connected by the second linker by non-covalent binding or covalent binding. Non-covalent binding includes p-p (aromatic) interactions, van der Waals interactions, H-bonding interactions, and ionic interactions. Preferably, the solid carrier and the capture moiety are connected by the first linker by non-covalent binding and/or the functional protein is connected to the capture moiety by the second linker by non-covalent binding. More preferably, the solid carrier and the capture moiety are connected by the first linker by non-covalent binding and/or the functional protein is connected to the capture moiety by the second linker by non-covalent binding, wherein the second linker is fused to the functional protein and binds non-covalently to the capture moiety.

In one embodiment, the solid carrier is selected from the group of organic particles, inorganic particles, organic-inorganic particles, self-assembling organic particles, silica particles, gold particles, titanium particles and is preferably a silica particle, more preferably a silica nanoparticle (SNP). The particle size is usually measured by measuring the diameter of the particles. In case the solid carrier is a monodisperse particle, the size is usually between 1 nm and 1000 μm, preferably between 10 nm and 100 μm, particularly between 50 nm and 50 μm, more particularly between 100 nm and 1 μm. In case the solid carrier is a polydisperse particle, the size is usually between 1 nm and 1000 μm, preferably between 10 nm and 100 μm, particularly between 50 nm and 50 μm.

Usually monodisperse particles or polydisperse particles, preferably monodisperse particles are used as solid carrier in the present invention. In a preferred embodiment, the monodisperse particles are spherical monodisperse particles. In a further preferred embodiment, the polydisperse particles are non-spherical polydisperse particles.

The solid carrier is usually provided in suspension. Suspension of the solid carrier can be, e.g., in water or buffer, preferably in buffer. Buffers which can be used in the method of the present invention are phosphate, piperazine-N,N′-bis(2-ethanesulfonic acid), 2-Hydroxy-3-morpholinopropanesulfonic acid, N,N-bis[2-hydroxyethyl]-2-aminoethanesulfonic acid), (3-(N-morpholino)propanesulfonic acid), 2-[[1,3-dihydroxy-2-(hydroxymethyl)propan-2-yl]amino]ethanesulfonic acid, 4-(2-hydroxyethyl)-1-piperazineethanesulfonic acid), 3-(N,N-Bis[2-hydroxyethyl]amino)-2-hydroxypropanesulfonic acid, N,N-Bis(2-hydroxyethyl)-3-amino-2-hydroxypropanesulfonic acid, N-[Tris(hydroxymethyl)methyl]glycine, Diglycine, 4-(2-Hydroxyethyl)-1-piperazinepropanesulfonic acid, N,N-Bis(2-hydroxyethyl)glycine, N-[Tris(hydroxymethyl)methyl]-3-aminopropanesulfonic acid, N-(1,1-Dimethyl-2-hydroxyethyl)-3-amino-2-hydroxypropanesulfonic acid. Preferably a phosphate buffer is used.

In one embodiment, the surface of the solid carrier as disclosed herein is modified to introduce a molecule or functional chemical group as anchoring point, i.e., as anchoring point for the first linker. Preferably, the anchoring point is an amine functional chemical group or moiety. As a non-limiting example, an amino-modified surface of the solid carrier, e.g., an amino-modified silica surface may be used as modified solid carrier. Such an amino-modified surface of the solid carrier may be obtained by reacting a solid carrier having a silica surface with an amino silane, e.g., with APTES. Preferably the surface of the solid carrier is only partially amino-modified. Thus in a preferred embodiment, the solid carrier is a solid carrier having a silica surface with an amino-modified surface, more preferably a solid carrier obtained by reacting the solid carrier having a silica surface with an amino silane, e.g., with APTES, even more preferably a solid carrier obtained by reacting the solid carrier having a silica surface partially with an amino silane, e.g., with APTES. Such a modified carrier may form an amide linkage between the first linker (e.g., Sulfo-NHS-Biotin) and the amine group at the surface of the carrier material. In one embodiment the introduced molecule or functional chemical group as anchoring point is homogeneously distributed on the surface of the solid carrier.

In one embodiment, the size ratio of solid carrier to functional protein is such that it allows binding of between 10 to 200, preferably of between 50 to 250, more preferably of between 20 to 150 protein molecules per particle.

In one embodiment, the thickness of the first protective layer is between 1 to 100 nm, 1 nm to 50 nm, 1 nm to 30 nm, 1 nm to 25 nm, 1 nm to 20 nm, or 1 nm to 15 nm.

In one embodiment, the thickness of the second protective layer is 1 to 100 nm, 1 nm to 50 nm, 1 nm to 30 nm, 1 nm to 25 nm, 1 nm to 20 nm, or 1 nm to 15 nm.

In some embodiments, the first and the second protective layer together have a defined thickness of about 1 nm to about 200 nm, usually 1 nm to about 100 nm, preferably about 1 nm to about 50 mm, more preferably about 1 nm to about 25 nm, even more preferably about 1 nm to about 20 nm, in particular about 1 nm to about 15 nm. The most preferred defined thickness is about 1 nm to about 15 nm. In some embodiments the first and the second protective layer together have a defined thickness of about 5 nm to about 100 nm, preferably about 5 nm to about 50 nm, more preferably about 5 nm to about 25 nm, even more preferably about 5 nm to about 20 nm, in particular about 5 nm to about 15 nm. The most preferred defined thickness is about 5 nm to about 15 nm.

In one embodiment, the first and the second protective layer consist of the same material. In a preferred embodiment, the first and the second protective layer consist of different material. The first and/or the second protective layer are usually porous and the pore size is between 1 nm and 100 nm, preferably between 1 nm and 20 nm.

The protective layer thickness can be measured, by using a microscope such as scanning electron microscope (SEM), transmission electron microscopy (TEM), scanning probe microscopy (SPM), light scattering methods or by ellipsometry.

The composition of the present invention is usually produced in a reaction vessel like a reactor. The formation of the first and/or second protective layer is usually carried out by forming the respective protective layer by building blocks, wherein the building blocks build the protective layer in a polycondensation reaction. The polycondensation can be effected in different solvents preferably in aqueous solution. Polycondensation can be easily controlled and stopped if appropriate, allowing achievement of a defined thickness of the protective layer. The choice of the building blocks, which can be used to build the second protective layer, may depend on the known structure of the functional protein in order to adapt the affinity of the second protective layer according to optimal and/or desired parameters. As building blocks for the first and/or second protective layer usually structural building blocks and protective building blocks are used to build the protective layer. Structural building blocks which can be used are, e.g., tetraethylorthosilicate (designated herein as “TEOS” or “T”). Protective building blocks which can be used are, e.g., 3-Aminopropyltriethoxysilane (designated herein as “APTES” or “A”), Propyltriethyoxysilane (designated herein as “PTES” or P”), Isobutyltriethoxysilane (designated as “IBTES”), Hydroxymethyltriethoxysilane (designated herein as “HTMEOS” or H), Benzyltriethoxysilane (designated herein as “BTES”), Ureidopropyltriethoxysilane (designated as “UPTES”), or Carboxyethyltriethoxysilane (designated herein as “CETES”). Structural building blocks are usually precursors of inorganic silica, capable of forming 4 covalent bonds in the layer formed. Protective building blocks, are usually organosilanes, bearing an organic moiety endowed with the ability to interact with the functional proteins (e.g., enzyme). Preferred structural building blocks are tetravalent silanes, in particular tetra-alkoxy-silanes. Preferred protective building blocks are trivalent silanes, in particular tri-alkoxy-silanes. More preferred structural building blocks are mixtures of tetravalent silanes and trivalent silanes, in particular, mixtures of tetra-alkoxy-silanes and tri-alkoxy-silanes. Even more preferred structural building blocks are selected from the group consisting of tetraethylorthosilicate, tetra-(2-hydroxyethyl)silane, and tetramethylorthosilicate. Even more preferred protective building blocks are selected from the group consisting of carboxyethylsilanetriol, benzylsilanes, propylsilanes, isobutylsilanes, n-octylsilanes, hydroxysilanes, bis(2-hydroxyethyl)-3-aminopropylsilanes, aminopropylsilanes, ureidopropylsilanes, (N-Acetylglycyl)-3-aminopropylsilanes, in particular selected from benzyltriethoxysilane, propyltriethoxysilane, isobutyltriethoxysilane, n-octyltriethoxysilane, hydroxymethyltriethoxysilane, bis(2-hydroxyethyl)-3-aminopropyltriethoxysilane, 3-Aminopropyltriethoxysilane, ureidopropyltriethoxysllane, (N-Acetylglycyl)-3-aminopropyltriethoxysilane, or selected from benzyltrimethoxysflane, propyltrimethoxysilane, isobutylimethoxysilane, n-octyltrimethoxysilane, hydroxyrnethyltrimethoxysilane, bis(2-hydroxyethyl)-3-aminopropyltrimethoxysilane, arninopropyltrimethoxysilane, ureidopropyltrimethoxysilane (N-Acetylglycyl)-3-aminopropyltrimethoxysilane or selected from benzyltrihydroxyethoxysilane, propyltrihydroxyethoxysilane, isobutyltrihydroxyethoxysilane, n-octyltrihydroxyethoxysilane, hydroxymefilyltrihydroxyethoxysilane, bis(2-hydroxyethyl)-3-aminopropyltrihydroxyethoxysilane, aminopropyltrihydroxyethoxysilane, Ureidopropyltrihydroxyethoxysilane (N-Acetylglycyl)-3-aminopropyltrihydroxymethoxysilane.

Particularly preferred building blocks are TEOS as structural building block and APTES, PTES and/or HTMEOS, preferably APTES and HTMEOS as protective building block. In particular, TEOS as structural building block and APTES and HTMEOS as protective building block are used to build the first protective layer and TEOS as structural building block and APTES as protective building block are used to build the second protective layer.

The reaction time of the building blocks with the solid carrier connected via the first linker to the capture moiety to form the first protective layer depends on the length of the first linker and the size of the capture moiety. Usually the reaction time is chosen so that the part of the capture moiety which bind the second linker is not covered by the first protective layer. The reaction is usually carried out for a time period of between 0.5 to 10 hours, preferably between 1 and 5 hours, more preferably between 1 and 4 hours, even more preferably between 2 and 4 hours, preferably in aqueous solution and preferably at room temperature of about 5° C. to about 15° C. or at about 10° C. The reaction time of the building blocks with the composition comprising the functional protein to form the second protective layer depends on the length of the second linker and the size of the functional protein. Usually the reaction time is chosen so that the part of the functional protein is not covered by the second protective layer. Alternatively, the reaction time is chosen so that the functional protein is fully covered by the second protective layer. The reaction is usually carried out for a time period of between 0.5 hour to 10 hours, preferably between 1 hour and 5 hours, more preferably between 1 hour and 4 hours, even more preferably between 1 hour and 2 hours, preferably in aqueous solution and preferably at room temperature of about 5° C. to about 15° C. or at about 10° C. The formation of the protective layer can be stopped by actively stopping the polycondensation reaction, e.g., by removing the non-reacted building blocks, e.g., by a washing step or by self-stopping of the polycondensation reaction caused by a limited amount of building blocks.

In one embodiment, the first linker is a tag, preferably, a tag selected from the group consisting of protein purification tags and affinity tags, more preferably, a tag selected from the group consisting of a Strep tag, a Twin-Strep tag®, biotin and a modified biotin, and even more preferably, a modified biotin, in particular, Sulfo-NHS-Biotin.

In one embodiment, the capture moiety is selected from the group consisting of an oligonucleotide; a polypeptide; an antibody or a functional fragment thereof; an avidin; an engineered avidin; a streptavidin; an engineered streptavidin, an aptamer; Spiegelmer™ (a L-RNA aptamer); a glutathione; or an S-peptide. Preferably, the capture moiety is selected from the group consisting of a polypeptide, an antibody or a functional fragment thereof, an avidin, an engineered avidin, a streptavidin and an engineered streptavidin. More preferably, the capture moiety is selected from the group consisting of an anti-biotin antibody a streptavidin and an engineered streptavidin, and is most preferably, an engineered streptavidin, in particular, Strep-Tactin®.

In one embodiment, the second linker is a tag, preferably, a tag selected from the group consisting of protein purification tags and affinity tags, more preferably, a tag selected from the group consisting of a Strep tag, a Twin-Strep tag®, biotin and a modified biotin, even more preferably, from the group consisting of Strep tag, a Twin-Strep tag®. Most preferably, the second linker is a Twin-Strep tag®, in particular, the second linker comprises or consists of an amino acid sequence comprising the sequence as shown in SEQ ID No: 1.

In a preferred embodiment, the functional protein is an enzyme, an antibody, or RNA which has catalytic activity enzyme or mixtures thereof, more preferably, an enzyme or a fragment thereof, even more preferably, an enzyme or a fragment thereof selected from the group consisting of oxidoreductases, transferases, hydrolases, lyases, isomerases, transpeptidases, or ligases or mixtures thereof. Particularly preferred is a hydrolase or a fragment thereof, more particular, a transpeptidase or a fragment thereof or a sequence-specific transpeptidase or a fragment thereof, even more particular, a sortase or a fragment thereof or a sequence-specific sortase or a fragment thereof. A preferred sortase is a sortase A3 or a fragment thereof, in particular a Sortase A from Staphylococcus aureus, or any functional fragment or mutant thereof, preferably a Sortase A variant having UniProt number KB—Q2FV99 (SRTA_STAA8) from Staphylococcus aureus (strain NCTC 8325).

In one embodiment, the second linker is bound to the functional protein, preferably covalently bound, more preferably fused to the functional protein, on the opposite side of the active site of the functional protein. Preferably, the second linker is bound, preferably, covalently bound, more preferably, fused to the functional protein, to the C terminus of the functional protein.

In one embodiment, the capture moiety is selected from the group consisting of streptavidin and a engineered streptavidin, preferably the capture moiety is Strep-Tactin®.

In one embodiment, the capture moiety contains binding sites for the first and second linkers, which are opposite to each other.

In one embodiment, the first protective layer fully embeds the solid carrier and the first linker and partially embeds the capture moiety.

Thus, in a preferred embodiment, the composition of the invention comprises a solid carrier; a capture moiety; a functional protein; a first linker connecting the capture moiety to the solid carrier; a second linker connecting the functional protein to the capture moiety; a first protective layer fully embedding the solid carrier, fully embedding the first linker, and partially embedding the capture moiety; a second protective layer partially embedding the capture moiety, fully embedding the second linker, and fully or partially embedding the functional protein.

In a more preferred embodiment, the composition of the invention comprises, a solid carrier; a capture moiety; a functional protein; a first linker connecting the capture moiety to the solid carrier; a second linker connecting the functional protein to the capture moiety; a first protective layer fully embedding the solid carrier, fully embedding the first linker, and partially embedding the capture moiety; a second protective layer partially embedding the capture moiety, fully embedding the second linker, and partially embedding the functional protein.

In a further preferred embodiment, the composition of the invention comprises, a solid carrier; a capture moiety; a functional protein; a first linker connecting the capture moiety to the solid carrier; a second linker connecting the functional protein to the capture moiety in a directional manner; a first protective layer fully embedding the solid carrier, fully embedding the first linker, and partially embedding the capture moiety; a second protective layer partially embedding the capture moiety, fully embedding the second linker, and fully or partially embedding the functional protein.

In a more preferred embodiment, the composition of the invention comprises, a solid carrier; a capture moiety; a functional protein; a first linker connecting the capture moiety to the solid carrier; a second linker connecting the functional protein to the capture moiety in a directional manner; a first protective layer fully embedding the solid carrier, fully embedding the first linker, and partially embedding the capture moiety; a second protective layer partially embedding the capture moiety, fully embedding the second linker, and partially embedding the functional protein.

In one embodiment, the capture moiety is fully embedded by the first and second protective layer.

In one embodiment, the capture moiety is fully embedded by the first and second protective layer so that the first protective layer embeds a part of the capture moiety and the second protective layer embeds the remaining part of the capture moiety.

Thus, in an even more preferred embodiment, the composition of the invention comprises, a solid carrier; a capture moiety; a functional protein; a first linker connecting the capture moiety to the solid carrier; a second linker connecting the functional protein to the capture moiety; a first protective layer fully embedding the solid carrier, fully embedding the first linker, and partially embedding the capture moiety; a second protective layer partially embedding the capture moiety, fully embedding the second linker, and fully or partially, preferably partially, embedding the functional protein, wherein the capture moiety is fully embedded by the first and second protective layer so that the first protective layer embeds a part of the capture moiety and the second protective layer embeds the remaining part of the capture moiety.

In a further even more preferred embodiment, the composition of the invention comprises, a solid carrier; a capture moiety; a functional protein; a first linker connecting the capture moiety to the solid carrier; a second linker connecting the functional protein to the capture moiety in a directional manner; a first protective layer fully embedding the solid carrier, fully embedding the first linker, and partially embedding the capture moiety; a second protective layer partially embedding the capture moiety, fully embedding the second linker, and fully or partially, preferably partially, embedding the functional protein, wherein the capture moiety is fully embedded by the first and second protective layer so that the first protective layer embeds a part of the capture moiety and the second protective layer embeds the remaining part of the capture moiety.

In one embodiment, the first protective layer embeds between 20% and 90%, preferably between 50% and 90%, more preferably between 60% and 95% of the capture moiety, wherein the section of the capture moiety where the second linker connects the functional protein to the capture moiety is not embedded by the first protective layer.

Preferably, the first protective layer fully embeds the solid carrier and the first linker and embeds between 20% and 90%, preferably between 50% and 90%, more preferably between 60% and 95% of the capture moiety, wherein the section of the capture moiety where the second linker connects the functional protein to the capture moiety is not embedded by the first protective layer.

In one embodiment, the first protective layer embeds between 20% and 90% of the capture moiety, wherein the section of the capture moiety where the second linker binds to the capture moiety to connect the capture moiety and the functional protein is not embedded.

In one embodiment, the second protective layer fully or partially embeds the capture moiety and fully embeds the second linker and embeds between 50% and 150%, usually embeds between 10% and 150%, preferably between 20% and 95% to 99%, more preferably between 30% and 90% to 95%, even more preferable between 60% and 90 to 99% of the functional protein.

In a further embodiment, the second protective layer fully or partially embeds the capture moiety and fully embeds the second linker and embeds between 20% and 95% to 99%, more preferably between 30% and 90% to 95%, even more preferable between 60% and 90 to 99% of the functional protein, wherein the active site of the functional protein is not embedded.

In one embodiment, the second protective layer fully embeds the first protective layer, partially embeds the capture moiety, fully embeds the second linker, and at least partially embeds the functional protein.

After the first protective layer has been formed, the solid carrier comprising the first linker and the capture moiety and the first protective layer can be stored. Storing is usually accomplished, e.g., by washing the composition formed, e.g., with a buffer and storing it suspended or solved in that buffer for a desired time period. In a preferred embodiment, the solid carrier comprising the first linker, the capture moiety, and the first protective layer is stored at a constant temperature between 2° C. to 25° C. In a further preferred embodiment, the solid carrier comprising the first linker, the capture moiety, and the first protective layer is stored 5 hours to 48 hours, preferably, 10 hours to 30 hours. More preferably, the solid carrier comprising the first linker, the capture moiety, and the first protective layer is stored at a constant temperature between 2° C. to 25° C., preferably, at room temperature for 10 hours to 30 hours.

In a further aspect, the present invention provides a method of producing a composition as described supra, e.g., a composition comprising a solid carrier; a capture moiety; a functional protein; a first linker connecting the capture moiety to the solid carrier; a second linker connecting the functional protein to the capture moiety; a first protective layer fully embedding the solid carrier, fully or partially embedding the first linker, and not or partially embedding the capture moiety; a second protective layer fully or partially embedding the capture moiety, fully embedding the second linker, and fully or partially embedding the functional protein; wherein the method comprises the steps of:
(a) connecting a capture moiety to a solid carrier by a first linker;
(b) forming a first protective layer on a surface of the solid carrier, the first protective layer fully embedding the solid carrier, fully or partially embedding the first linker, and not or partially embedding the capture moiety;
(c) connecting a functional protein to the capture moiety by a second linker; and
(d) forming a second protective layer on the surface of the first protective layer, the second protective layer fully or partially embedding the capture moiety, fully embedding the second linker, and fully or partially embedding the functional protein.

Step (a) is usually carried out by adding the first linker to the solid carrier in suspension in water or a buffer. The suspension can be stirred, e.g., at 400 rpm, 20° C. for 30 min. After an optional washing step and resuspension of particles the capture moiety is added and the resulting mixture is usually stirred, e.g., at 20° C., 400 rpm for 1 h.

The formation of a first protective layer on the surface of the solid carrier fully embedding the solid carrier, fully or partially embedding the first linker and not or partially embedding the capture moiety according to step (b) is usually carried out by adding a building block, e.g., a tetravalent silane like TEOS to the mixture obtained with step (a) and allowed to react, e.g., at 10° C., 400 rpm for, e.g., 1 h. A further building block, e.g., a trivalent silane like APTES or two trivalent silanes, e.g., APTES and hydroxymethyltriethoxysilane are then added to the previous mixture and reacted for, e.g., 4 hours. Samples of the composition covered by the first protective layer can be taken at various times during reaction to obtain compositions with first protective layers of different thicknesses. The samples can be stored, e.g., at r.t. for 24 h for curing.

The binding of the functional protein to the capture moiety according to step (c) is usually carried out by adding the functional protein, e.g., the enzyme to the samples obtained after step (b). The obtained suspension is then usually stirred, e.g., at 20° C., 400 rpm for 1 h. Subsequently and with or without intermediate washing, a building block, e.g., a tetravalent silane like TEOS is added to the particles and allowed to react, e.g., at 10° C., 400 rpm for 1 h. A further building block, e.g., a trivalent silane like APTES is then added to the mixture. Samples of the composition covered by the second protective layer can be taken at various times during reaction to obtain compositions with second protective layers of different thicknesses. The samples can be stored, e.g., at r.t. for 24 h for curing.

In one embodiment, the composition obtained after step (b) is stored before binding the functional protein to the capture moiety in step (c). In a further embodiment, the composition is stored after step (d). In a preferred embodiment, the storage occurs at a constant temperature between 2° C. to 25° C. In a further preferred embodiment, the storage occurs for 5 hours to 48 hours, preferably 10° C. to 25 hours.

In one embodiment, the second protective layer fully embeds the first protective layer.

In one embodiment, the first and the second protective layer consist of the same material or different material, preferably consist of different material.

In a further aspect, the present invention provides a method of producing an immunoligand/payload conjugate, which method comprises conjugating (e.g., carrying out a conjugation reaction of) a payload to an immunoligand by means of the composition as described supra.

In one embodiment, the functional protein of the composition is a transpeptidase or a fragment thereof, preferably a sequence-specific transpeptidase or a fragment thereof. In one embodiment, the transpeptidase or the fragment thereof of the composition of the present invention catalyzes the conjugation reaction.

In a further embodiment, the composition comprising a transpeptidase or a fragment thereof is incubated with the immunoligand and the payload.

In a further embodiment, the composition comprising a transpeptidase or a fragment thereof is
i) provided in a solution;
ii) the solution comprising the composition is incubated with the immunoligand and the payload; and
ii) after the conjugation reaction has taken place, the solution is centrifuged and a supernatant containing an immunoligand/payload conjugate is removed.

In one embodiment, the payload and/or the immunoligand either
a) consists, entirely, of a protein or a peptide, or
b) comprises at least one protein or peptide domain, or
c) comprises at least one protein as the immunoligand and a small molecular weight molecule as the payload, or
d) comprises at least one protein as the immunoligand and a small molecular weight toxin as the payload, or
e) comprises at least one protein or peptide chain and wherein, further, the at least one protein or peptide chain comprises an amino acid sequence that is detectible by the transpeptidase, or a fragment thereof.

In one embodiment, the immunoligand comprised in the immunoligand/payload conjugate is selected from the group consisting of an antibody, a modified antibody format, an antibody derivative or a fragment, and/or an antibody mimetic.

In one embodiment, the payload comprised in the immunoligand/payload conjugate is selected from the group consisting of a marker, a tag, and a drug, preferably is a small molecular weight drug or small molecular weight toxin.

In one embodiment, the payload comprised in the immunoligand/payload conjugate is selected from the group consisting of a marker, a tag, and a drug, wherein the marker is selected from the group consisting of a radiolabel, preferably a radioactively labelled peptide or protein, a fluorescent label, preferably a fluorescent peptide or protein, and an enzyme label, preferably a peroxidase.

In one embodiment, the payload comprised in the immunoligand/payload conjugate is a marker selected from the group consisting of a radiolabel, preferably a radioactively labelled peptide or protein, a fluorescent label, preferably a fluorescent peptide or protein, and an enzyme label, preferably a peroxidase.

In one embodiment, the payload comprised in the immunoligand/payload conjugate is selected from the group consisting of a marker, a tag, and a drug, wherein the drug is selected from the group consisting of a cytokine, a radioactive agent, an anti-inflammatory drug, a toxin, and a chemotherapeutic agent.

In one embodiment, the payload comprised in the immunoligand/payload conjugate is a drug selected from the group consisting of a cytokine, a radioactive agent, an anti-inflammatory drug, a toxin, and a chemotherapeutic agent.

In a further aspect, the present invention provides an immunoligand/payload conjugate obtained by the method as described supra.

### EXAMPLES

Materials: Tetraethyl orthosilicate (T, ≥99%), (3-aminopropyl)triethoxysilane (A, ≥98%), N-Hydroxysulfosuccinimide sodium salt (Sulfo-NHS), ammonium hydroxide (NH4OH, ACS grade, 28-30%), ethanol (EtOH, ACS grade, anhydrous), glutaraldehyde (grade I, 25% in water), N,N′-Dicyclohexylcarbodiimide (DCC, 99%), Trizma® base (>99.9%), CH3CN and glycerol were purchased from Sigma-Aldrich (Switzerland). n-propyltriethoxysilane (P, 97%), and Hexahydro-2-oxo-1H-thieno[3,4-d]imidazole-4-pentanoic acid (Biotin, 97%) were purchased from ABCR (Germany). Hydroxymethyltriethoxysilane (H, 50% in ethanol) was purchased from Gelest (USA). Precast protein gels (4-20% Mini-PROTEAN® TGX™ Precast Protein Gels, 15-well, 15 μl) and Ladder (Precision Plus Protein™ Dual Xtra Prestained Proteins Standards) were purchased from Bio-Rad. Potassium phosphate salts and TFA were purchased from Fisher Scientific (Switzerland). Sodium phosphate salts, HEPES salt, NaCl, CaCl₂), glycine, and DTT were purchased from Carl Roth (Germany). Gly5-modified Fluorescein isothiocyanate (FITC) was purchased from Bachem (Switzerland). Sortase A (SrtA) was purchased from AGC Biologics. Strep-Tactin lyophilized was purchased from IBA Lifesciences (Germany). Aeroperl 300 particles were purchased from Evonik. LPQTG-tagged monoclonal antibodies were purchase from Evitria/WuXi Biologics. Gly2-modified toxin was purchased from Levena Biopharma. PBS was purchased from BioConcept.

### Example 1: Optimization of Unspecific Coupling of Sortase a Enzyme on the Particles

SNPs were produced using the conventional Stöber method adapted from the report of Imhof et al. (J. Phys. Chem. B 1999, 103, 1408), as follows. Ethanol (345.4 ml), ammonia 25% (39.3 ml) and TEOS (tetraethylorthosilicate, 15.3 ml) were mixed in a round bottom flask and this mixture was stirred at 600 rpm during 20 hours, at a constant temperature of 20° C. The resulting precipitate was subsequently washed twice with ethanol and twice with water, and freeze-dried to yield bare SNPs that were characterized using scanning electron microscopy (Zeiss, SUPRA 40 VP). The acquired micrographs were used for particle size measurement using the analysis® (Olympus) software package (statistical analysis carried out on 100 measurements).

In order to introduce amine functional chemical groups at the surface of the SNPs allowing immobilization of functional proteins or, in this case, sortase A enzyme, the SNPs were reacted with an amino-silane. It is important to note that this modification should only be partial in order to leave silanol groups for the further attachment of the protective layer. For this SNPs in suspension in water (20 mL; 10 mg/ml) were incubated with APTES (3-aminopropyltriethoxysilane, 33 mg) during 90 minutes at 20° C. After two washing steps in water, the resulting amino-modified SNPs (SNPs-NH2) were obtained.

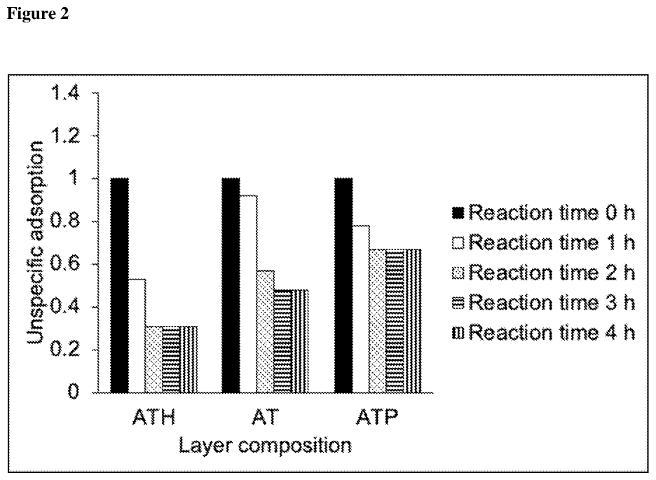

The biotin-modification of amino-modified nanoparticles (SNPs-NH2) was performed using the water-soluble biotinylation reagent Sulfo-NHS-Biotin. In a typical procedure, Sulfo-NHS-biotin was added to amino modified silica particles (SNPs-NH2) in phosphate buffer at pH 8.0. The suspension was stirred at 400 rpm, 20° C. for 30 min. After washing and resuspension of the biotinylated particles (SNPs-bio) (3.2 mg/mL, 15 mL) in phosphate buffer pH 8.0, Strep-Tactin (500 μg/mL) was added, and the resulting mixture (SNPs-bio-ST) was stirred at 20° C., 400 rpm for 1 h. Subsequently and without intermediate washing, 43.3 μL of tetraethyl orthosilicate (T) were added to the particles (3.2 mg/mL, 14.5 mL) and allowed to react at 10° C., 400 rpm for 1 h. Then, particles were incubated with three different organosilanes mixtures containing: 3-aminopropyl-triethoxysilane (A), hydroxymethyltri-ethoxysilane (H), and propyltriethoxysilane (P). Consequently, three different layers (ATH, AT and ATP) were produced and compared. In more details, for the synthesis of the layer made of ATH, 16.5 μL of A and 27.1 μL of H were added to the suspension of particles; for the synthesis of the AT layer, 28.2 μL of T and 6.7 μL of A were added and for the ATP layer 17.5 μL of T, 6.7 μL of A and 11.3 μL of P were added and samples were collected every hour for 4 hours. The different samples of SNPs-bio-ST-AT or SNPs-bio-ST-ATH or SNPs-bio-ST-ATP were stored at room temperature for 24 h. A layer of around 1.4 nm was obtained for all the three different compositions. Around 50% of Strep-Tactin (the capture moiety) was covered by the layer. Then, sortase A having UniProt number KB—Q2FV99 (SRTA_STAA8) from Staphylococcus aureus (strain NCTC 8325) fused to a Twin-Strep Tag (SEQ ID No:1) at the C-terminus (130 μg/mL) was added to the three samples of particles carrying biotin-Strep-Tactin and the suspension was stirred at 20° C., 400 rpm for 1 h. The layers made of ATH, AT and ATP allowed a decrease of the non-specific adsorption of sortase A up to 69%, 51% and 32%, respectively (FIG. 2). Non-specific adsorption results from intermolecular forces (including ionic interactions and van der Waals forces) between the available surface of the carrier and the Sortase A. The chemical functionalities of the used organosilanes (A, H, and P) modified the chemical charge at the surface of the particles decreasing the non-specific adsorption of Sortase A. Non-specific adsorption causes the uncontrolled binding of Sortase A at the surface of the carrier and consequently a decrease of bioconjugation.

### Example 2: Directional Immobilization of Sortase a and Shielding

On the basis of the results obtained in example 1, the layer with a composition ATH presented the lowest unspecific adsorption of sortase A. Consequently, the system including the particles (3.2 mg/mL, 2.5 mL) carrying Strep-Tactin shielded with a layer made of ATH was selected as carrier for the immobilization of sortase A in a directional manner. Sortase A having UniProt number KB—Q2FV99 (SRTA_STAA8) from Staphylococcus aureus (strain NCTC 8325) fused to a Twin-Strep Tag (SEQ ID No:1) at the C-terminus (130 μg/mL) was added and the suspension was stirred at 20° C., 400 rpm for 1 h and subsequently, and without intermediate washing, 12 μL of tetraethyl orthosilicate (T) were added to the particles (3.2 mg/mL, 2.5 mL) and allowed to react at 10° C., 400 rpm for 1 h. 3-aminopropyl-triethoxysilane (A) (2.83 μL) was then added to the previous mixture. 625 μL were collected every hour for 4 hours. In particular, after 3-hours incubation with A and T the layer was 9.8 nm and after 4-hours the layer was 11.2 nm.

### Example 3: Bioconjugation Reaction

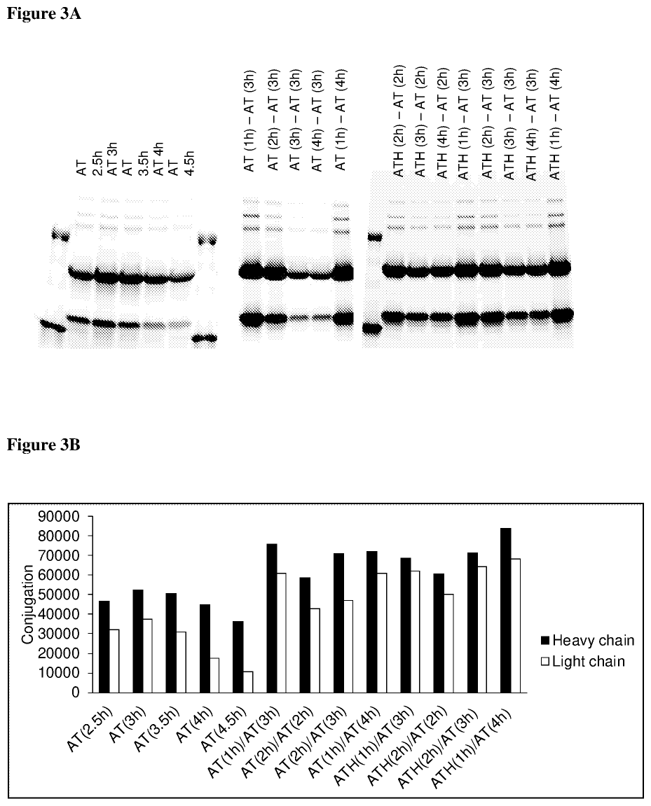

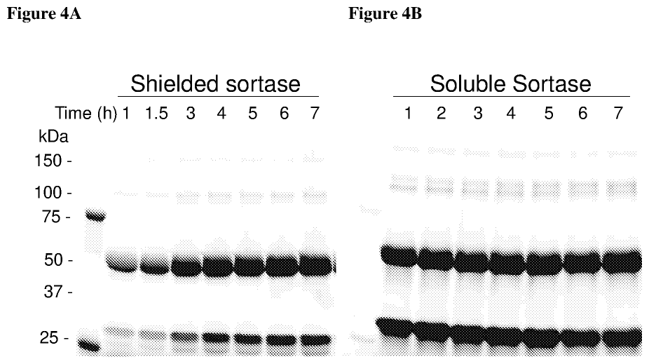

The bioconjugation reaction catalyzed by sortase-SNPs or by soluble sortase enzyme was measured by a conjugation of a recombinant antibody with sortase recognition motifs present at the C-termini of the antibody heavy and light chains to a penta-glycine-modified fluorescein isothiocyanate (Gly5-FITC), both of which are therefore substrates for a sortase A mediated transpeptidation reaction. Sortase A having UniProt number KB—Q2FV99 (SRTA_STAA8) from Staphylococcus aureus (strain NCTC 8325) fused to a Twin-Strep Tag (SEQ ID No:1) at the C-terminus was immobilized in a directional manner via a Strep-Tactin capture moiety present on the surface of silica particles and shielded with two different layers (AT-AT and ATH-AT) or with a protective single layer (AT) according to the procedure as described in Example 2. The kinetics of bioconjugation using a system in which sortase A (UniProt number KB—Q2FV99 (SRTA_STAA8) from Staphylococcus aureus (strain NCTC 8325)) fused to a Twin-Strep Tag (SEQ ID No:1) at the C-terminus was immobilized on the surface of silica particles in a directional manner and shielded with two different layers (AT-AT and ATH-AT) was unexpectedly higher compared to a system in which sortase A was directly coupled to SNPs and shielded with a single protective single layer (AT). In a typical bioconjugation experiment, a solution of sortase immobilized on silica particles and shielded, was incubated with an antibody (mAb) (10 μM) and Gly₅-FITC (200 μM) in Tris-Buffer pH 7.5, at 25° C., 800 rpm for 4 h in the dark. After conjugation reaction, the samples were centrifuged, and the supernatants were transferred into empty vials. The supernatants of each samples were analysed by sodium-dodecyl-sulphate polyacrylamide gel electrophoresis (SDS-PAGE) (FIG. 3a) and the bioconjugation efficiencies of each systems were determined (FIG. 3b).

When Sortase A is covalently anchored on the surface of amino-modified silica particles using glutaraldehyde as cross-linker, and partially shielded with a layer of 2 nm, the bioconjugation of antibody to the probe is the lowest: AT(2.5 h); AT(3 h); AT(3.5 h); AT(4 h); AT(4.5 h). When Sortase A is anchored onto silica particles in a directional manner on SNPs already carrying a capturing moiety and carrying a double layer (AT/AT or AT/ATH) higher values of antibody bioconjugation are obtained. The best value is obtained with a double layer prepared with AT(1h)/ATH(4h).

### Example 4: Comparison of Bioconjugation Reactions Catalyzed by a Soluble Sortase a and a Directionally Immobilized and Shielded Sortase a

Bioconjugation reactions catalyzed by a soluble Sortase A and a directionally immobilized and shielded Sortase A (synthesized as shown in Example 2) were performed as follows: 10 μM of the recombinant antibody used in Example 3 and 200 μM of Gly5-FITC were added to a suspension of immobilized and shielded sortase A (3 μM) in Tris buffer (50 mM, 150 mM NaCl, 5 mM CaCl₂), pH 7.5) and shaken in a thermomixer in the dark at 25° C., 800 rpm for 7 h. Aliquots were collected every hour. In order to stop the bioconjugation reaction, the suspensions were centrifuged, and the supernatants were transferred into new tubes. Similarly, 3 μM of soluble Sortase A were incubated with 10 μM of antibody and 200 μM of Gly5-FITC. Fluorescence scan showed that the activity of conjugation of the shielded sortase was identical to the soluble enzyme.

### Example 5: Site-Specific Immobilization of Sortase a on Aeroperl 300 Particles and Shielding

Commercially available porous silica particles (diameter 20-60 μm) were modified to anchor the sortase A. In more details, Aeroperl particles in suspension in water (20 mL; 10 mg/ml) were incubated with APTES (3-aminopropyltriethoxysilane, 33 mg) during 90 minutes at 20° C. After two washing steps in water, the resulting amino-modified Aeroperl (Aer-NH2) were obtained. The biotin-modification of amino-modified particles (Aer-NH2) has been performed using the water-soluble biotinylation reagent Sulfo-NHS-Biotin.

In a typical procedure, Sulfo-NHS-biotin was added to amino modified silica particles (Aer-NH2) in phosphate buffer pH 8. The suspension was mixed at 1000 rpm, 20° C. for 30 min. After washing and resuspension of the biotinylated particles (Aer-bio) (1.6 mg/mL, 25 mL) in phosphate buffer pH 8, Strep-Tactin (500 μg/mL) was added, and the resulting mixture (Aer-bio-ST) was mixed at 20° C., 1000 rpm for 1 h. Subsequently and without intermediate washing, 37 μL of tetraethyl orthosilicate (T) were added to the particles (1.6 mg/mL, 23.9 mL) and allowed to react at 10° C., mixing at 1000 rpm for 1 h. Then, particles were incubated with an organosilanes mixtures containing: 3-aminopropyl-triethoxysilane (A) and hydroxymethyl-triethoxysilane (H). In more details, for the synthesis of the layer made of ATH, 14.1 μL of A and 23.1 μL of H were added to the suspension of particles and the mixture was allowed to react for 1 h at 10° C. mixing at 1000 rpm. Around 50% of Strep-Tactin (the capture moiety) was covered by the layer. The sample of Aer-bio-ST-ATH was stored at room temperature for 24 h for curing. Then, sortase A having UniProt number KB—Q2FV99 (SRTA_STAA8) from Staphylococcus aureus (strain NCTC 8325) fused to a Twin-Strep Tag (SEQ ID No:1) at the C-terminus (130 μg/mL) was added to the sample of particles carrying biotin-Strep-Tactin and the suspension was mixed at 20° C., mixing at 1000 rpm for 1 h. Subsequently, and without intermediate washing, 57.4 μL of tetraethyl orthosilicate (T) were added to the particles (1.6 mg/mL, 23.9 mL) and allowed to react at 20° C., mixing at 1000 rpm for 1 h. 3-Aminopropyl-triethoxysilane (A) (13.5 μL) was then added to the previous mixture and let to react at 20° C., 4 h mixing at 1000 rpm.

Bioconjugation: Immobilized-Sortase-Mediated Antibody Conjugation

Payloads were conjugated to mAbs by incubating LPQTG-tagged monoclonal antibodies (10 μM) with Gly2-modified toxin (400 μM) in the presence of 3 M immobilised Sortase A in 50 mM HEPES, 150 mM NaCl, 5 mM CaCl₂), 10% glycerol at pH 7.5 16-20 h at 25° C. The immobilized Sortase A conjugated samples were centrifuged at 200 rcf for 5 min. The supernatant of the ISMAC were collected in a separate tube. The remaining Sortase beads were washed 3 times with 50 mM NaH₂PO₄, 100 mM NaCl, pH 8 and pooled with the ISMAC supernatant. The ISMAC was purified by passing it through a Gravity flow Protein A column (GE Healthcare, #28-9852-54) equilibrated with 24 column volumes (CV) of 25 mM HEPES, 150 mM NaCl, 10% glycerol (v/v), pH 7.5. The column was then washed with 24 CV of same buffer. Bound conjugate was eluted with elution buffer (100 mM glycine, 50 mM NaCl, 10% glycerol (v/v), pH 2.7) as fractions collected into tubes containing 1:25 1 M HEPES, pH 8.0 to neutralize the solution. Protein containing fractions were pooled and formulated in Phosphate buffered saline (PBS) using desalting/formulation columns (Zeba Spin column, ThermoFisher, #89892, 5 mL resin) according to the manufacturer's instructions. The formulated ADCs were filtered with a 0.22 μm PES syringe filter unit and corresponding disposable syringe.

ADC Analytics

The drug loading was assessed by Reverse Phase Chromatography (RP-LC) on a PLRP-S 1000 Å, 2.1×50 mm, 3 μm column (Agilent, #PL1912-3301), run at 0.7 mL/min at 60° C. with a 9 min linear gradient between 0.1% TFA/3.0% CH₃CN/96.9% H₂O and 0.1% TFA/99.9% CH₃CN. Samples were first reduced by incubation with DTT at 37° C. for 15 min and centrifuged for 5 min at 14000 rcf.

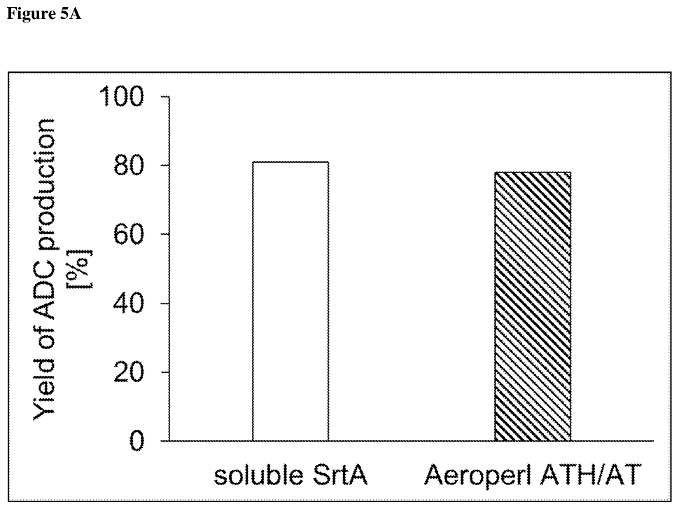

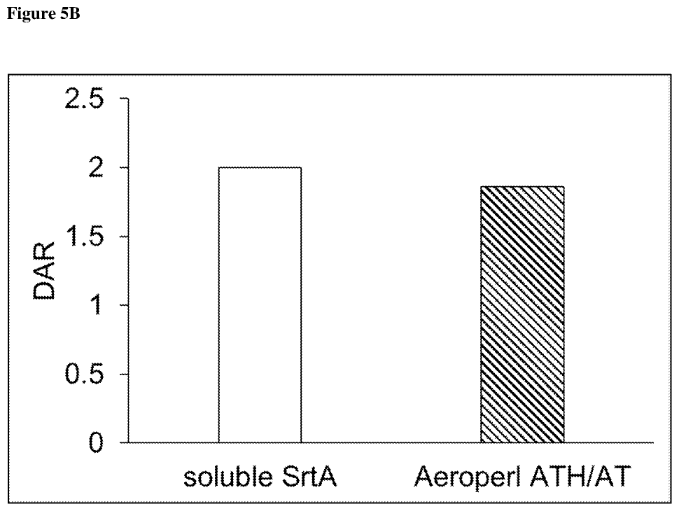

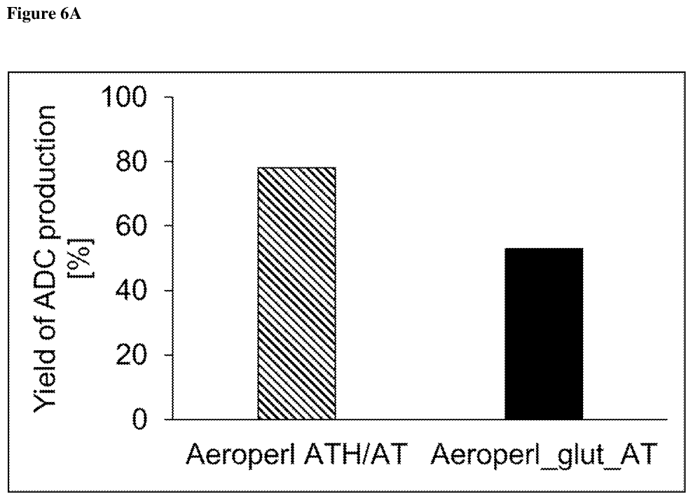

When Sortase A is anchored onto Aeroperl particles carrying a double layer (ATH/AT), the obtained yield of ADC production and the DAR value are comparable to the ones obtained with the soluble Sortase A. In more details, the Aeroperl ATH/AT showed a yield of ADC production of 78% and the soluble enzyme showed a yield of ADC production of 81% (FIG. 5A). Additionally, the Aeroperl ATH/AT showed a DAR of 1.86 and the soluble enzyme showed a DAR value of 2.00 (FIG. 5B).

### Example 6: Non Site-Specific Immobilization of Sortase a on Aeroperl 300 Particles and Shielding (Comparative Experiment)

Commercially available porous silica particles (diameter 20-60 μm) were modified to anchor the sortase as described in WO2015/014888. In more details, Aeroperl particles in suspension in water (20 mL; 10 mg/ml) were incubated with APTES (3-aminopropyltriethoxysilane, 33 mg) during 90 minutes at 20 C. After two washing steps in water, the resulting amino-modified Aeroperl (Aer-NH2) were obtained.

The glutaraldehyde-modification of amino-modified particles (Aer-NH2) has been performed using the glutaraldehyde (25% in H₂O). In a typical procedure, glutaraldehyde was added to amino modified silica particles (Aer-NH2) in water. The suspension was mixed at 1000 rpm, 20° C. for 30 min. After washing and resuspension of the glutaraldehyde particles (Aer-glut) (3.2 mg/mL, 15 mL) in phosphate buffer pH 8, SortaseA (45 μg/mL) was added, and the resulting mixture (Aer-glut-SortaseA) was mixed at 20° C., 1000 rpm for 1 h. Subsequently and without intermediate washing, 36 μL of tetraethyl orthosilicate (T) were added to the particles (3.2 mg/mL, 7.5 mL) and allowed to react at 10° C., mixing at 1000 rpm for 1 h. Then, particles were incubated with 3-aminopropyl-triethoxysilane (A). In more details, for the synthesis of the layer made of AT, 8.48 μL of A were added to the suspension of particles and the mixture was allowed to react for 3 h at 10° C. mixing at 1000 rpm. The sample of Aer-bio-ST-ATH was stored at room temperature for 24 h for curing.

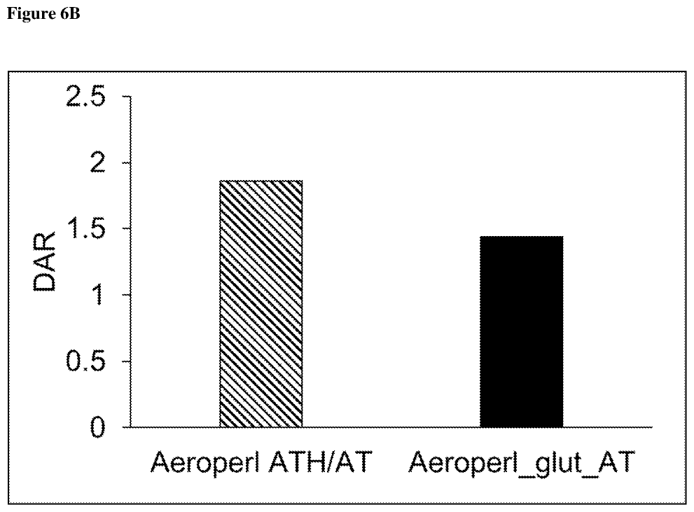

Immobilized-Sortase-Mediated Antibody Conjugation and ADC analytics has been carried out as described in example 5. When Sortase A is anchored onto Aeroperl particles using glutaraldehyde and shielded with a layer (AT) as described in WO2015/014888, the yield of ADC production and the DAR values are lower than the ones obtained with the site specific immobilized Sortase A as described in Example 5. In more details, the immobilized through glutaraldehyde and shielded (Aeroperl_glutaraldehyde_AT) showed a yield of ADC production of 53% and the site-oriented immobilized Sortase A showed a yield of ADC production of 78% (FIG. 6A). Additionally, the Aeroperl_glutaraldehyde_AT showed a DAR of 1.44 and the site-oriented immobilized Sortase A showed a DAR value of 1.86 (FIG. 6B).

### Incorporation of Sequence Listing

Incorporated herein by reference in its entirety is the Sequence Listing for the above-identified Application. The Sequence Listing is disclosed on a computer-readable ASCII text file titled “Sequence_Listing_2268-33_PCTUS.txt”, created on Aug. 17, 2023. The sequence.txt file is 1 KB in size.

1. A composition comprising:
a solid carrier;
a capture moiety;
a functional protein;
a first linker connecting the capture moiety to the solid carrier;
a second linker connecting the functional protein to the capture moiety;
a first protective layer fully embedding the solid carrier, fully or partially embedding the first linker, and not or partially embedding the capture moiety; and
a second protective layer fully or partially embedding the capture moiety, fully embedding the second linker, and fully or partially embedding the functional protein.

2. The composition according to claim 1, wherein the functional protein is an enzyme or a fragment thereof.

3. The composition according to claim 1, wherein the functional protein is a transpeptidase or a fragment thereof.

4. The composition according to claim 1, wherein the functional protein is a sortase or a fragment thereof.

5. The composition according to claim 1, wherein the first linker and/or second linker is a tag selected from the group consisting of protein purification tags and affinity tags.

6. The composition according to claim 1, wherein the first linker is selected from the group consisting of a Strep-Tag, a Twin-Strep tag®, biotin, and a modified biotin.

7. The composition according to claim 1, wherein the second linker is selected from the group consisting of a Strep-Tag, a Twin-Strep tag®, biotin, and a modified biotin.

8. The composition according to claim 1, wherein the capture moiety is selected from the group consisting of a streptavidin and an engineered streptavidin.

9. The composition according to claim 1, wherein the first protective layer fully embeds the solid carrier, fully embeds the first linker, and partially embeds the capture moiety, and the second protective layer partially embeds the capture moiety, fully embeds the second linker, and fully or partially embeds the functional protein.

10. A method of producing a composition according to claim 1, wherein the method comprises:
(a) connecting a capture moiety to a solid carrier by a first linker;
(b) forming a first protective layer on a surface of the solid carrier, the first protective layer fully embedding the solid carrier, fully or partially embedding the first linker, and not or partially embedding the capture moiety;
(c) connecting a functional protein to the capture moiety by a second linker; and
(d) forming a second protective layer on the surface of the first protective layer, the second protective layer fully or partially embedding the capture moiety, fully embedding the second linker, and fully or partially embedding the functional protein.

11. A method of producing an immunoligand/payload conjugate, wherein the method comprises carrying out a conjugation reaction of a payload to an immunoligand by means of a composition according to claim 3.

12. The method according to claim 11, wherein the transpeptidase or the fragment thereof of the composition catalyzes the conjugation reaction.

13. The method according to claim 11, wherein the method comprises incubating the composition with the immunoligand and the payload.

14. The method according to claim 11, wherein the method comprises:
i) providing the composition in a solution;
ii) incubating the solution comprising the composition with the immunoligand and the payload; and
ii) after the conjugation reaction has taken place, centrifuging the solution and removing a supernatant containing an immunoligand/payload conjugate.

15. The method according to claim 11, wherein the payload and/or the immunoligand either
a) consists, entirely, of a protein or a peptide, or
b) comprises at least one protein or peptide domain, or
c) comprises at least one protein as the immunoligand and a small molecular weight molecule as the payload, or
d) comprises at least one protein as the immunoligand and a small molecular weight toxin as the payload, or
e) comprises at least one protein or peptide chain and wherein, further, the at least one protein or peptide chain comprises an amino acid sequence detectable by the transpeptidase, or a fragment thereof.

16. The method according to claim 14, wherein the immunoligand comprised in the immunoligand/payload conjugate is selected from the group consisting of an antibody, a modified antibody format, an antibody derivative or a fragment, and/or an antibody mimetic.

17. The method according to claim 14, wherein the payload comprised in the immunoligand/payload conjugate is selected from the group consisting of a marker, a processing tag, and a drug.

18. An immunoligand/payload conjugate obtained by a method according to claim 14.


SEQUENCE LISTING (2 sequences)

SEQ ID NO: 1 - protein, 47 aa, Artificial Sequence
  Second linker
KIFVATEVKLEWSHPQFEKGGGSGGGSGGSAWSHPQFEKGSHHHHHH

SEQ ID NO: 2 - protein, 8 aa, Artificial Sequence
  Twin-Strep tag
WSHPQFEK